(19) United States
(12) Patent Application Publication
(10) Pub. No.: US 2005/0084483 A1
(43) Pub. Date: Apr. 21, 2005

(54) Farm animal product with probiotic enterococcus bacteria

(75) Inventors: Jane A.Z. Leedle, Hartland, WI (US); William P. Kautz, Waukesha, WI (US); Steven C. Johnson, Waukesha, WI (US); Kelly F. Lechtenberg, Oakland, NE (US)

Correspondence Address: HUNTON & WILLIAMS LLP, Suite 1200, 1900 K Street, N.W., Washington, DC, 20006-1109, US

(73) Assignee: CHR. HANSEN A/S, Hoersholm (DK)

(21) Appl. No.: 10/902,292

(22) Filed: Jul. 30, 2004

Related U.S. Application Data
(60) Provisional application No. 60/490,943, filed on Jul. 30, 2003.
(60) Provisional application No. 60/497,564, filed on Aug. 26, 2003.

(30) Foreign Application Priority Data
Aug. 26, 2003 (EP) 03077658.7

(51) Int. Cl. A61K 45/00; A23K 1/165; A23K 1/17

(52) U.S. Cl. 424/93.45; 424/442

(57) ABSTRACT

A farm animal product comprising probiotic Enterococcus bacteria and the use of this product to reduce the number of pathogenic Escherichia coli O157:H7 cells in farm animals such as cattle.

### CROSS-REFERENCE TO RELATED APPLICATIONS

This application claims the benefit of U.S. Provisional Application No. 60/490,943, filed Jul. 30, 2003, and U.S. Provisional Application No. 60/497,564, filed Aug. 26, 2003, and claims priority to EP Application No. 03 077 658.7, filed Aug. 26, 2003. All of the foregoing applications are herein incorporated by reference in their entireties.

### FIELD OF THE INVENTION

The present invention relates to a farm animal product comprising probiotic Enterococcus bacteria and the use of this product to reduce the number of pathogenic Escherichia coli O157:H7 cells in farm animals such as cattle.

### BACKGROUND OF THE INVENTION

The animal feed industry, such as the beef cattle industry, is experiencing challenges like never before, and one of the most critical challenges to the industry is food safety. For example, the consumer and governmental agencies are requiring that beef sold in restaurants, grocery stores and meat markets be as safe and pathogen free as possible. Meat packing companies are looking to the feed yards and the cattle producers to implement strategies to help achieve this goal.

The demand for food industry control of potentially contaminating pathogens starts, as noted, at the consumer level, who are stating, through their buying patterns at the meat case, that they need a product in which they can have confidence. Subsequently, retailers look to wholesalers and the packing companies. The packers are looking to the feed yards and, e.g., the cattle producers to take the necessary steps to help reduce this problem by adopting safety standards and procedures at all points along the production chain. This issue is key and it will take adjustments of management procedures by all entities involved in beef production to address this issue.

It has been well documented through scientific and medical research that the predominant organism at the root of food safety issues is Escherichia Coli (E. coli) O157:H7, otherwise known as enterohaemorrharic E. coli microorganism. E. coli O157:H7 is one of hundreds of strains of the bacterium. Although most strains are harmless and live in the intestines of healthy humans and animals, this strain produces a powerful toxin and can cause severe illness. It also possesses other significant attributes, which contribute to its ability to cause disease. One of the more notable of its characteristics is the size of the infectious dose, which is incredibly small in comparison with those for most other food-borne pathogens. Figures as low as two bacteria per 25 g food have been quoted capable of creating a disease condition.

The strain E. coli O157:H7 was first recognized as a cause of illness in 1982 during an outbreak of severe bloody diarrhea; the outbreak was traced to contaminated hamburgers. Since then, most infections have resulted from eating undercooked ground beef. The combination of letters and numbers in the name of the bacterium refers to the specific markers found on its surface and distinguishes it from other types of E. coli. Another pathogen of concern includes strains of Salmonella, with both E. coli and Salmonella commonly existing in the gastrointestinal tracts of cattle. These organisms are endemic and commonly found in virtually all phases of production. While they may not cause a problem in the host animal they can cause illness and even death in humans. Cattle become “infected” with this organism through exposure in their natural environment. After the organism is ingested it travels to the intestine where it adheres to the tract lining. Meat is “contaminated” by the organism during the slaughtering and processing stages when intestinal contents can come in contact with other meat surfaces and subsequently become mixed with ground beef.

In humans, an E. coli infection can lead to bloody diarrhea and even kidney failure. In some persons, particularly children under 5 years of age and the elderly, the infection can also cause a complication called hemolytic uremic syndrome, in which the red blood cells are destroyed and the kidneys fail. About two to seven percent of infections lead to this complication. In the United States, hemolytic uremic syndrome is the principal cause of acute kidney failure in children, and most cases of hemolytic uremic syndrome are caused by the strain E. coli O157:H7.

Most illnesses have been associated with eating undercooked, contaminated ground beef. In addition, however, person-to-person contact in families and childcare centers is also an important mode of transmission. Infection can also occur after drinking raw milk and after swimming in or drinking sewage-contaminated water. As an example of this, the USDA Food Safety and Inspection Service (FSIS) has estimated that consumption of meat contaminated with pathogenic bacteria annually results in thousands of deaths and millions of illnesses in the U.S. alone. The government estimates the annual losses in production and medical costs may reach as high as $35 billion. The problem is well documented and identified.

Having recognized this problem and a need to solve it or at least diminish it, recent proactive efforts have been shown by the industry. The proactive efforts exerted by, e.g., the US beef industry have resulted in recommendations of expanded research and accelerated use of intervening methodologies by industry leaders. Control and treatment techniques such as the irradiation of beef products post slaughter, use of new vaccines in cattle and direct feeding of certain additives are all under serious investigation and consideration as contributing solutions. Of these, the use of feed additives has gained significant interest, largely due to simplicity of administration.

One particular group of feed additives showing significant promise in this area is probiotic or Direct-Fed Microbial (“DFM”) products. The use of DFM's has grown significantly over recent years largely as a means of enhancing the health and performance of the animal. The use of bacterial-based DFM's in ruminant diets for specific applications has become widely recognized. Products of this nature often contain lactobacilli with Lactobacillus acidophilus being one of the most common.

Most bacterial-based DFM's are beneficial because they have effects in the lower gut and not in the rumen. For example, Lactobacillus acidophilus produces lactic acid, which may lower the pH in small intestines to levels that inhibit the growth of pathogenic microbes, one of the reasons for the current interest. Early research with DFM in ruminants first involved applications for young calves fed milk, calves being weaned, or cattle being shipped. These animals, in many cases, are highly stressed or had a microbial gut ecosystem that was not fully mature. Young cattle have immature digestive tracts that are obviously more prone to upset by pathogenic bacteria. Cattle that are shipped are often on limited feed and water for prolonged periods of time during transit. During these periods microbial populations may decrease in numbers, thus resulting in digestive tracts that are in less than optimal condition. Large doses of beneficial organisms were thought to re-colonize a stressed intestinal environment and return gut function to normal.

The American Meat Institute (“AMI”) Foundation published in 2002 a result of a research study that was done by Mindy Brashears and Michael Galyean of Texas Tech University. At the filing date of the present application the study was published on the Internet at the address: http://www.amif.org/ProbioticsReport042302.pdf. According to the study, the feeding of two different Lactobacillus acidophilus bacteria strains gave a significant reduction (P<0.05) in the incidence of E. Coli O157:H7 in the faeces of finishing cattle. The experimental design of the study was:
Control—Cattle fed with a standard diet,
NP 747—Cattle fed with a standard diet with 1×10⁹CFU Lactobacillus acidophilus strain NPC 747 mixed in water and added to the diet at the time of feeding;
NP 750—Cattle fed with a standard diet with 1×10⁹CFU Lactobacillus acidophilus strain NPC 750 mixed in water and added to the diet at the time of feeding.
   The result of the study was explained as: “Just 14 d after initiating treatment, significant (P<0.05) differences were observed among the three treatment groups. At this sampling time 56.6% of the control animals were positive, whereas only 20% of the animals fed with the NPC 747 sample and 11% of those fed with the NPC 750 probiotic were positive. Comparing the data based on a positive pen basis, significant (P<0.05) differences were also observed. Forty-one percent of the pens receiving the NPC 750 treatment had at least one positive animal, which was significantly (P<0.05) the percentage of pens in cattle receiving NPC 747 (66% with at least one positive sample).” Expressed in log units, the best data reduces the number of positives by around 0.5 logs (1 log is a ten times reduction).

Ongoing work has shown that levels of E. coli increase in cattle during the finishing period. Feeding of specific strains of beneficial bacteria has shown to reduce the levels of pathogenic proliferation. Studies of this nature are eliciting positive responses from a number of meat packers. Many packers are making strong recommendations to their supplying feed yards to feed probiotics to help with this issue. Their position is that if the level of E. coli entering the facility via the animal is reduced, their ability to further reduce contamination is vastly improved.

The research into this area is ongoing by universities and a number of companies. In particular several bacterial strains developed by Lallemand Animal Nutrition (“LAN;” Milwaukee, Wis.) have shown significant results in reducing the concentrations of E. coli O157:H7 and Salmonella via a process known as competitive exclusion. Competitive exclusion is a process by which beneficial bacteria are used to colonize the lining of the intestinal walls, reducing the area available for attachment by pathogenic microbes. The results so far confirm earlier theories that part of the effect noted through the feeding of beneficial bacteria results from this reduction in the area of the intestinal lining available to the pathogen for attachment.

Certain specific strains of Lactobacilli and Propionibacterium developed by LAN have proven effective at reducing the numbers of these pathogens under different environmental conditions. Probiotic research has shown the effectiveness of gut colonization of beneficial bacteria in reducing pathogenic populations through competitive exclusion of these harmful organisms. In recent in vitro collaborative work by LAN and AgTech (Waukesha, Wis., a 15 year-old biotech research company), it was found that several bacterial strains were highly effective in inhibiting the growth and development of strains of Salmonella and E. coli including E. coli O157:H7. The results indicate that, in particular, the BG2FO4 strain of Lactobacillus acidophilus was very effective in inhibiting all strains of pathogenic E. coli tested. It is also important to note that the inhibition was a result of not only competitive exclusion but also of the action of extracellular bacteriocins produced by the Lactobacillus. The results also indicated inhibition of several strains of Salmonella. A concluding result was that Lactobacillus acidophilus BG2FO4 exhibits a high degree of pathogen oriented anti-microbial activity and is an excellent choice for use in beef cattle for this purpose.

U.S. Pat. No. 5,718,894 (“the '894 patent”) describes a formulation for use in the promotion of growth or weight gain in a farm animal. The '894 patent states that the formulation comprises two groups of bacteria: a so-called first bacterium capable of producing lactic acid in the gastrointestinal tract of the animal; and a second bacterium capable of producing a bactericide to which the bacteria are resistant, wherein said second bacterium is a Bacillus. The '894 patent states that bactericide produced by the Bacillus strain is capable of combating microorganisms that are the positive agent of enteric disorders, e.g. Staphylococcus aureus, E. coli and Salmonella (see column 2, lines 23-31). The '894 patent states that examples of bacterium capable of producing lactic acid are bacteria of the genus Lactobacillus or Enterococcus. According to the '894 patent, they are distinguished by their ability to produce lactic acid and thus reduce the local pH in the gastrointestinal tract of the animal (see column 2, lines 8-22). A specific formulation for use in pigs is described in the '894 patent. It is composed of the four strains Lactobacillus, Enterococcus faecalis, Enterococcus faecium and Bacillus licheniformis. The '894 patent states that each strain is used at 10⁹ cfu/g.

In summary, current research has already revealed and continues to reveal useful methodologies for the control of pathogenic bacterial populations in farm animals such as beef cattle. In relation to use of probiotic bacteria, relevant detailed studies have mainly focused on use of suitable Lactobacillus acidophilus strains.

### SUMMARY OF THE INVENTION

The problem to be solved by the present invention includes the provision of a composition (also referred to herein as “farm animal product”) that has an improved ability with respect to decreasing the number of Escherichia coli O157:H7 in farm animals (preferably cattle) when the farm animals are challenged with Escherichia coli O157:H7 pathogen.

One solution to this problem is that the present inventors have found that a composition comprising Enterococcus strains works better than a corresponding composition comprising similar amounts (CFU/g) of Lactobacillus acidophilus strains.

Working examples herein demonstrate, inter alia, that cattle fed with about 109 CFU Enterococcus bacteria per day had a significant reduction of the number of Escherichia coli O157:H7 quantified in the faeces of the challenged animals. The reduction was about 1.5 to 2 logs in 10 to 14 days. 1 log unit denotes a ten times reduction and 2 log units denotes a 100 times reduction.

In the Brashears & Galyean study discussed supra, a corresponding example of an animal feed composition comprising Lactobacillus acidophilus resulted in around 0.5 log reduction. Without being limited by theory, it is believed that direct-fed microbial (“DFM”) products currently being commercially sold in cattle to reduce the number of Escherichia coli are based on Lactobacillus acidophilus, and they reduce the number of the target pathogen by only 0.5 logs.

In relation to the reduction of the number of Escherichia coli O157:H7, Enterococcus bacteria work better than Lactobacillus acidophilus bacteria. However, in one embodiment of the present invention, apart from Enterococcus, the farm animal product may comprise smaller amounts of Lactobacillus acidophilus bacteria than those of Enterococcus bacteria.

Accordingly, one aspect of the invention relates to a farm animal product comprising at least 10⁶ CFU/g of probiotic Enterococcus bacteria, characterized in that, if the product contains Lactobacillus acidophilus, the product has at least 2.5 times more of Enterococcus bacteria than Lactobacillus acidophilus bacteria measured as CFU/g.

Another aspect of the invention relates to a method of feeding a farm animal comprising feeding the farm animal with a farm animal product comprising at least 10⁶ CFU/g of probiotic Enterococcus bacteria, characterized in that, if the product contains Lactobacillus acidophilus, the product has at least 2.5 times more of Enterococcus bacteria than Lactobacillus acidophilus bacteria measured as CFU/g.

A further aspect of the invention relates to a method of feeding a farm animal comprising feeding the farm animal with a farm animal product comprising at least 10⁶ CFU/g of probiotic Enterococcus bacteria, wherein said product reduces the number of Escherichia coli O157:H7 cells quantified in faeces of challenged animals by at least 1.5 logs.

### DETAILED DESCRIPTION OF THE INVENTION

As used herein, the term “probiotic” is a well-defined term in the art and relates to a class of microorganisms defined as live microbial organisms that confer health benefit to farm animal hosts or is not pathogenic to same. The beneficial effects include improvement of the microbial balance of the intestinal micro flora and the improvement of the properties of the indigenous micro flora when, for example, the microorganism is orally administered.

As used herein, the term “Enterococcus” is a well-known and well-defined term for this Enterococcus genus of bacteria species. For further details see, e.g., the standard reference book Bergeys Manual of Systematic Bacteriology. Based on the general knowledge of one of ordinary skill in the art, the skilled person is capable of determining whether or not a specific Enterococcus bacterium of interest is a bacterium of the Enterococcus genus.

In all embodiments of the invention, when the term “comprising” or “including” is used, the respective description also includes the same composition, product or item which consists essentially of or consists of the ingredients of the composition, product or item.

As used herein, the term “CFU” denotes Colony Forming Units. The term “CFU/g” refers to CFU/g of farm animal product.

Farm Animal Product

In one embodiment of the present invention, the farm animal product comprises suitable farm animal feedstuff ingredients in addition to an Enterococcus bacteria. The skilled person is aware of selecting the adequate ingredients in relation to the specific farm animal of interest. Herein, such suitable farm animal feedstuff ingredients may be termed farm animal feedstuff ingredients known per se or farm animal feedstuff ingredients.

In this aspect of the invention, these ingredients should be in concentrations adjusted to meet animal's dietary requirements and may include nutrient ingredients such as animal protein products, at about 0—about 95 weight percent; plant protein products, at about 0—about 95 weight percent; poultry egg products, at about 0—about 25 weight percent.

In a further embodiment of the invention, the farm animal product may also comprise other suitable ingredients such as antibiotics such as Sarafin, Romet, Terramycin at about 0.01—about 50 weight percent; cyanocobalamin at about 40—about 60 mg/kg; D-biotin at about 5—about 20 mg/kg; D-pantothenic acid at about 250—about 350 mg/kg; folic acid at about 10—about 30 mg/kg; L-ascorbyl-2-polyphosphate (STAY-C, stable form of vitamin C) at about 1,000—about 4,000 mg/kg; myo-inositol at about 3,000—about 4,000 mg/kg; niacin at about 600—about 800 mg/kg; p-amino-benzoic acid at about 350—about 450 mg/kg; pyridoxine hydrochloride at about 40—about 60 mg/kg; riboflavin at about 125—about 175 mg/kg; thiamine hydrochloride at about 50—about 80 mg/kg; choline chloride at about 6,500—about 7,500 mg/kg.

In another embodiment of the invention, the farm animal product may be present in any suitable form, such as a powder, liquid or in form of pellets or tablets.

One embodiment of a farm animal product is a composition comprising Enterococcus bacteria in a bolus, or, in a further embodiment, in a gelatin bolus. A particular embodiment of a farm animal product is a composition comprising Enterococcus bacteria, Glucidex IT12 (about 30%) and Type 4A Act Molecular Sieve Powder (about 10%).

In another embodiment of the invention, the farm animal product may be in form of, e.g., two different compositions. Such an embodiment includes one composition comprising the suitable farm animal feedstuff ingredients and the other composition comprising the Enterococcus bacteria as described herein. In a further aspect of the invention, the farm animal product is comprised of such two compositions and accompanied by suitable instructions to administer them to the farm animal either simultaneously or sequentially. In other words, in an embodiment of the present invention, while the farm animals are fed with the suitable farm animal feedstuff ingredients, they should also be fed with the probiotic Enterococcus bacteria-containing product as described herein.

In an alternative embodiment of the invention, the farm animal product may be in form of a composition comprising the suitable farm animal feedstuff ingredients and the probiotic Enterococcus bacteria as described herein. In a particular embodiment of the invention, the composition is in the form of a suitable powder, a liquid or in the form of pellets or tablets.

In another embodiment of the invention, in order to improve some stability aspects of the probiotic bacteria, it may be advantageous to provide the farm animal product as a stable emulsion of solids in-oil comprised of lipid soluble bioactive compounds such as inhibitory furanones dissolved in lipid forms of the continuous phase and with dry feed ingredients and the probiotic bacteria of interest forming the dispersed phase of the stable emulsion. See, e.g., WO 02/00035 for further details.

In a further embodiment of the present invention, the farm animal product may be in form of a capsule, e.g., a microencapsulated product.

As described supra, one preferred embodiment of the invention is to use Enterococcus bacteria instead of Lactobacillus acidophilus bacteria.

In an embodiment of the invention, the farm animal product comprises at least 5 times more of Enterococcus bacteria than Lactobacillus acidophilus bacteria measured as CFU/g, in a further embodiment comprises at least 50 times more of Enterococcus bacteria than Lactobacillus acidophilus bacteria measured as CFU/g, in another embodiment comprises at least 500 times more of Enterococcus bacteria than Lactobacillus acidophilus bacteria measured as CFU/g and in another further embodiment comprises at least 5000 times more of Enterococcus bacteria than Lactobacillus acidophilus bacteria measured as CFU/g.

In other words, one embodiment of a farm animal product does not comprise measurable amounts of Lactobacillus acidophilus. Such an embodiment of a farm animal product may be denoted a farm animal product consisting essentially of probiotic Enterococcus bacteria, or consisting essentially of at least 10 CFU/g of probiotic Enterococcus bacteria. In another embodiment, such an embodiment of a farm animal product may be denoted a farm animal product consisting of probiotic Enterococcus bacteria, or consisting of at least 10⁶ CFU/g of probiotic Enterococcus bacteria. Another embodiment of a farm animal product does not comprise significant amounts of Lactobacillus acidophilus. In all embodiments including Enterococcus bacteria, one or more strains of such bacteria may be present.

In one embodiment of the present invention, the farm animal product comprises the probiotic Enterococcus bacteria in a concentration of at least. 10⁶ CFU/g, in a further embodiment comprises the probiotic Enterococcus bacteria in a concentration of at least 10⁸ CFU/g, in a further embodiment comprises the probiotic Enterococcus bacteria in a concentration of at least 10¹⁰ CFU/g and in a further embodiment comprises the probiotic Enterococcus bacteria in a concentration of at least 10¹¹ CFU/g. In embodiments of the invention, the farm animal product comprises the probiotic Enterococcus bacteria in a concentration of less than 10¹⁴ CFU/g. In an embodiment of the invention, the farm animal product comprises the probiotic Enterococcus bacteria in a concentration from 10⁸ CFU/g to 10¹² CFU/g. In another embodiment of the invention, the farm animal product comprises the probiotic Enterococcus bacteria in a concentration of approximately 2×10¹¹ CFU/g. In a further embodiment of the invention, the farm animal product comprises the probiotic Enterococcus bacteria in a concentration of approximately 5×10⁹ CFU/g. In a further embodiment, the farm animal product comprises probiotic Enterococcus bacteria in a concentration of approximately 5×10¹⁰ CFU/g. In another embodiment of the present invention, the farm animal product comprises probiotic Enterococcus bacteria in a concentration of from approximately 5×10⁹ CFU/g to approximately 2×10¹¹ CFU/g. In another embodiment of the invention, the farm animal product comprises probiotic Enterococcus bacteria in a concentration of from 10⁶ CFU/g to approximately 2×10¹¹ CFU/g.

Probiotic Enterococcus Bacteria

The probiotic Enterococcus bacteria described herein may be any probiotic Enterococcus bacteria. Based on the information disclosed herein, the skilled person is capable of selecting a specific Enterococcus strain of interest.

In embodiments of the invention, the Enterococcus strain is an Enterococcus faecium.

In an embodiment of the present invention, Enterococcus faecium strains are SF-273 (CHCC 4202) and SF-301 (CHCC 3828). In a further embodiment, the farm animal product comprises both SF-273 (CHCC 4202) and SF-301 (CHCC 3828), and, in a further embodiment the two strains are present, in a ratio of 50:50 (based on potency). In another embodiment, the two strains are present in the farm animal product in a ratio of 60:40 or 40:60. Enterococcus faecium strain number SF-273 was deposited on Apr. 14, 1998, under ATCC 27273, in the American Type Culture Collection, 10801 University Blvd., Manassas, Va. 20110-2209. Also, SF-273 was deposited in the Chr Hansen Culture Collection under CHCC 4202. Enterococcus faecium strain number SF-301 was deposited on Nov. 27, 1997, under DSM 4789, in the German Culture Collection, Deutsche Sammlung von Mikoorganismen und Zellkulturen GmbH, Mascheroder Weg 1b, D-38124 Braunschweig, Germany. Also, SF-301 was deposited in the Chr Hansen Culture Collection under CHCC 3828.

In an additional embodiment of the present invention, the Enterococcus bacteria are selected to be tolerant of the following conditions: high acid (pH 4.0), high concentrations of volatile fatty acids (200 to 400 mM mixtures of acetic, propionic and butyric acids) and complete anaerobiosis.

Working Example 2 herein sets forth an example of one preferred assay to test if an Enterococcus bacteria is tolerant to these conditions.

In another embodiment of the present invention, the Enterococcus bacteria are also oxygen scavengers. Such bacteria are much more stable to oxygen exposure, moisture and heat than L. acidophilus.

Based on the information provided herein, combined with the knowledge of the skilled artisan, it is routine work to select Enterococcus bacteria tolerant to the conditions provided supra.

In embodiments of the invention in which the product bacteria are tolerant to the conditions and/or are oxygen scavengers, such traits combined are useful to confer a high degree of survival within the gastrointestinal tract of feedlot cattle. In an embodiment of the invention, the product bacteria are metabolically active upon ingestion by the animal. This activity can have an immediate impact either on the environment within the gastrointestinal tract, or on the E. coli cells present or on the receptor sites/niches with which the E. coli cells associate. Or, in another embodiment of the invention, it could be any combination of these influences.

In accordance with the present invention, the product containing the Enterococcus bacteria can be in many different forms. In one embodiment of the present invention, the product containing the Enterococcus bacteria is a feed product. In another embodiment of the present invention, the product containing the Enterococcus bacteria is in the form of a direct dose to the animal. In an embodiment of the invention the product containing the Enterococcus bacteria is in the form of a direct dose to the animal through a stomach tube.

Farm Animal

In an aspect of the present invention, the farm animal may be a pig, a cow, a cattle, a sheep, a chicken, a duck or an ostrich. In another embodiment of the invention, the farm animal is a ruminant animal, in particular a cattle or a cow. In a further embodiment of the invention, the farm animals are cattle.

A method for Administering a Farm Animal Product as Described Herein to a Farm Animal

The feeding may be done according to the art, and the skilled artisan is aware of how to properly feed farm animals.

In an embodiment of the present invention, the farm animals are fed with an amount of farm animal product that provides at least 10⁷ CFU Enterococcus bacteria per animal per day, in another embodiment the farm animals are fed with an amount of farm animal product that provides at least 10⁸ CFU Enterococcus bacteria per animal per day, in another embodiment the farm animals are fed with an amount of farm animal product that provides at least 10⁹ CFU Enterococcus bacteria per animal per day and in another embodiment the farm animals are fed with an amount of farm animal product that provides at least 10¹⁰ CFU Enterococcus bacteria per animal per day. In embodiments of the invention, the farm animals are fed with an amount of farm animal product that provides less than 10¹³ CFU Enterococcus bacteria per animal per day. In an additional embodiment of the invention, the farm animals are fed with an amount of farm animal product that provides approximately 2×10¹¹ CFU Enterococcus bacteria per animal per day. In another embodiment, the farm animals are fed with an amount of farm animal product that provides approximately 5×10⁹ CFU Enterococcus bacteria per animal per day. In a further embodiment of the invention, the farm animals are fed with an amount of farm animal product that provides approximately 5×10¹⁰ CFU Enterococcus bacteria per animal per day. In a further embodiment of the invention, the farm animals are fed with an amount of farm animal product that provides from 10⁸ CFU to 10¹¹ Enterococcus bacteria per animal per day, in a further embodiment the farm animals are fed with an amount of farm animal product that provides from 10⁹ CFU to 10¹⁰ Enterococcus bacteria per animal per day. In a further embodiment of the invention, the farm animals are fed with an amount of farm animal product that provides from approximately 5×10⁹ CFU to approximately 2×10¹¹ CFU Enterococcus bacteria per animal per day.

In an embodiment of the invention, the animals are fed once a day. In an alternative embodiment of the invention, they may be fed twice a day or, in another embodiment of the invention, once every second day. The skilled artisan is aware of what is best in relation to a specific farm animal of interest.

In another embodiment of the present invention, the farm animals are fed with the farm animal product as described herein at least once a day for at least 10 days, and in a further embodiment the farm animals are fed with the farm animal product as described herein at least once a day for at least 20 days.

In a further embodiment of the invention, the farm animals are fed with the farm animal product as described herein until slaughter. In a particular embodiment, the farm animals are fed with the farm animal product as described herein at least once a day for at least the last 10 days until slaughter, and in another embodiment the farm animals are fed with the farm animal product as described herein at least once a day for at least the last 20 days until slaughter.

In a further embodiment of the present invention, the farm animals are fed with the farm animal product as described herein for about 26 days. In an additional embodiment of the present invention, the farm animals are fed for about 26 days with an amount of farm animal product that provides from approximately 5×10⁹ CFU to approximately 2×10¹¹ CFU Enterococcus bacteria per animal per day. In an additional embodiment of the present invention, the farm animals are fed for about 26 days with an amount of farm animal product that provides from approximately 5×10⁹ CFU to approximately 5×10¹⁰ CFU Enterococcus bacteria per animal per day. In an additional embodiment, the farm animals are fed for about 26 days prior to slaughter with an amount of farm animal product that provides from approximately 5×10⁹ CFU to approximately 5×10¹⁰ CFU Enterococcus bacteria per animal per day. Additionally, in this embodiment, the animals can be fed the farm animal product set forth in Example 3.

In another embodiment of the invention, the farm animal product is used for feeding the animals in an amount and for a number of days where the farm animal product reduces the number of Escherichia coli O157:H7 cells quantified in the faeces of the challenged animals by at least 1.5 logs, or, in another embodiment, by at least 2 logs. In another embodiment of the present invention, the farm animal product reduces the number of Escherichia coli O157:H7 cells quantified in the faeces of the challenged animals by at least 1.5 logs in the first few days of treatment, or, in another embodiment, by at least 2 logs in the first few days of treatment. In another embodiment of the present invention, the farm animal product reduces the number of Escherichia coli O157:H7 cells quantified in the faeces of the challenged animals by at least 1.5 logs in 3 days of treatment, or, in another embodiment, by at least 2 logs in 3 days of treatment. In another embodiment of the present invention, the farm animal product reduces the number of Escherichia coli O157:H7 cells quantified in the faeces of the challenged animals by at least 1.5 logs in 10 to 14 days of treatment, or, in another embodiment, by at least 2 logs in 10 to 14 days of treatment. In yet another embodiment of the present invention, the farm animal product reduces the number of Escherichia coli O157:H7 cells quantified in the faeces of the challenged animals by about 1.5 logs to about 2 logs. In yet another embodiment of the present invention, the farm animal product reduces the number of Escherichia coli O157:H7 cells quantified in the faeces of the challenged animals by about 1.5 logs to about 2 logs within 3 days of treatment. Additionally, in this embodiment, the animals can be fed the farm animal product set forth in Example 3.

Working examples herein describe a suitable assay to quantitatively measure this.

In accordance with the present invention, the product containing the Enterococcus bacteria can be administered to the animal in many ways. In one embodiment of the present invention, the product containing the Enterococcus bacteria is fed to the animal as a feed product. In another embodiment of the present invention, the product containing the Enterococcus bacteria is dosed directly to the animal. In an embodiment of the invention, the product containing the Enterococcus bacteria is dosed directly to the animal through a stomach tube.

The entire disclosure of each document cited (including patents, patent applications, journal articles, abstracts, laboratory manuals, books, or other disclosures) in the Background of the Invention, Summary, Detailed Description, and Examples is herein incorporated by reference.

### EXAMPLES

### Example 1

### Dose Titration Study to Determine the Effect of Two Different Dosing Regimens of a Characterized Direct Fed Microbial Culture on the Post-Challenge Fecal Shedding of Escherichia coli O157:H7

Bacteria Containing Products:

CHB DFM: Two Enterococcus faecium strains present together at 50:50 (based on potency) in a gelatin bolus. The strains are SF-273 (CHCC 4202) and SF-301 (CHCC 3828). The two Enterococcus faecium strains each are present in the product at 2.5×10⁹ CFU/g.

CHB Probios TC: A corresponding product, which comprises probiotic Enterococcus faecium bacteria in a similar CFU/g as for CHB DFM. This product also comprised active dry yeast at around 2.5×10⁹ CFU/g.

Objective:

The objective of this project was to explore the effect of two different doses of CHB DFM on the magnitude of fecal shedding of Nal^{r} E. coli O157: H7 in beef cattle fed a highly fermentable complete ration.

Materials and Methods:

Twelve beef steer calves weighing approximately 400 pounds were ranked by body weight and randomly allotted to one of three treatment groups. The treatment groups were control (no DFM) and DFM dosed at 2, or 20 g/head/day. All calves were orally dosed with DFM using gelatin boluses in order to assure that they received their daily target dose of DFM.

A similar strategy was used for Probios TC, and Probios TC was dosed at 2, or 20 g/head/day.

Cattle were housed together in a single isolation room and allowed to commingle throughout the study. All cattle were allowed to consume a ration formulated with rolled corn and corn gluten feed containing sodium monensin (30 grams/ton). Fecal samples were collected and provided to detection of E. coli O157: H7 by enrichment technique to assure that calves were free of (Nal^{r}) E. coli O157: H7 prior to challenge.

Prior to the initiation of the study, all cattle were identified using duplicate unique Temple® ear tags and were vaccinated with a modified live virus vaccine containing IBR, BVD, P13, and BRSV (Bovishield 4®, Pfizer), 2 ml intranasal vaccination containing IBR and P13 (TSV-2®, Pfizer), a single 2 ml injection of a Clostridial vaccine (Vision-7®, Intervet), and a single injection of anthelmintic to control internal and external parasites (Dectomax®, Pfizer).

Experimental Design:

The following treatment groups were evaluated:
Treatment A: Non-Medicated Control (n=4)
Treatment B: CHB—Direct Fed Microbial (CHB-DFM) at a rate of 2 grams/head/day (n=4)
Treatment C: CHB—Direct Fed Microbial (CHB-DFM) at a rate of 20 grams/head/day (n=4)
Treatment D: Probios TC at a rate of 2 grams/head/day
Treatment E: Probios TC at a rate of 20 grams/head/day

Animals were inoculated with E. coli O157:H7, strains FRIK 1123 and FRIK 2000 which were adapted to nalidixic acid (Nal^{r}) in the laboratory (20 g/ml). The organisms were grown in GN broth (Difco laboratories, Detroit, Mich.) for 7 h (approx. 0.8 abs at 600 nm), the two cultures were pooled and colony counts of the pooled cultures were done by spread plate technique. Each animal was inoculated (day 0) by using a stomach tube through a Frick speculum with 60 ml of the pooled cultures containing 8.6×10⁸ CFU/ml of Nal^{r} E. coli O157:H7 (5.2×10¹⁰ CFU/animal). Following administration of the challenge material, the tube was flushed with 120 mL of sterile phosphate buffered saline.

Animals were evaluated daily for evidence of adverse reactions. Fecal (rectal) specimens were collected on 1, 3, 5, 8, 10, 12, 14, 17, 19, 23, 24, 26, and 30 days following oral challenge. Fecal samples were placed in whirl packs, packed in ice and provided for detection and quantification of Nal^{r} E. coli O157: H7.

Detection and Enumeration of Nal^{r} E. coli O157:H7:

One gram of faeces was added to 9.0 ml of GN broth containing 50 μl (0.05 mg/liter) of cefixime (C), 200 μl (10 mg/liter) of cefsulodin (C), and 100 μl (8 mg/liter) of vancomycin (V). Samples were vortexed for 30 sec, serially diluted, and 100 μl of 10⁻¹, 10⁻², 10⁻³ dilutions was spread plated, in triplicate, onto sorbitol MacConkey agar (SMAC) plates containing μg/ml of NaI. The remaining GN broth was incubated as an enrichment step in the isolation procedure. After 6 h incubation at 37° C., 1.0 ml was transferred into 9.0 ml of GNccv broth and incubated an additional 18 to 24 h at 37° C. The inoculated SMAC plates were incubated for 24 h at 37° C. and typical sorbitol-negative (gray colored) colonies were counted. A maximum of three colonies per sample per animal were collected, streaked onto blood agar plates, and incubated for 24 h at 37° C. The indole test was done on colonies from the blood agar plates; indole positive colonies were tested for agglutination specific for O157 (Oxoid Diagnostic Reagents, Basingstock, Hampshire, England).

If E. coli O157:H7 colonies were not detected by direct plating (detection limit>10²/g), GNccv broth incubated for 18 to 24 h was plated, in duplicate, on SMAC plates containing NaI (μg/ml) and incubated for 24 h at 37° C. Following incubation, three colonies per sample with typical colony morphology (from the enriched samples) were streaked on blood agar plates and incubated for 24 h at 37° C. The indole test was done on colonies from the blood agar plates and indole positive colonies were tested for agglutination specific for O157.

Statistical Analysis:

The study had two outcomes of interest. First, the level of fecal E. coli O157:H7 shedding was compared between treatment groups. The independent variables were treatment, day, and the treatment by day interaction. The comparison was done using repeated measures analysis of variance (MIXED procedure, SAS Institute Inc.). The outcome was the level of fecal shedding (CFU/g of faeces) with group as the treatment and day as the repeated measure. Day and treatment interaction was included as a fixed effect. Colony counts were log transformed prior to analysis.

Results:

Cattle in all groups shed at least 102 CFU/g (range 102 to 104) of Nal^{r} E. coli O157:H7 in the faeces during the first week (days 1, 3 and 5) after inoculation. After that there was a general decrease in magnitude of shedding and numbers of Nal^{r} E. coli O157:H7 recovered ranged from 10² to undetectable (see Table 1). After day 12, the shedding pattern was somewhat erratic with concentrations fluctuating from 10² to undetectable. Treatment groups fed DFM at 2 or 20 g shed lower concentrations of Nal^{r} E. coli O157:H7 the faeces compared to the control (P values=0.01 and 0.06, respectively). The extent of reduction was greater in the group dosed with 20 g compared to 2 g dose. Table 3 reports data from a similar study made during another period than the study behind the data of Table 1. This second data confirms the overall results described above.

Conclusions

DFM at 20 g per animal per day caused a significant reduction in the level of shedding of Nal^{r} E. coli O157:H7.

TABLE 1
Effects of Direct-fed microbials (DFM) on fecal shedding of
Nalidixic acid-resistant E. coli O157:H7 in cattle.

| | |
|---|---|
| Sampling | DFM, g/animal/day |

| days | 0 | 2 | 20 |
|---|---|---|---|
| 0 | 3.5 × 10⁴ | 3.5 × 10⁴ | 3.5 × 10⁴ |
| 1 | 2.5 × 10⁴ a | 1.5 × 10⁴ a | 3.5 × 10² a |
| 3 | 1.5 × 10⁴ a | 9.1 × 10³ a | 2.3 × 10² a |
| 5 | 6.5 × 10³ a | 1.6 × 10³ a | 2.2 × 10³ a |
| 8 | 7.6 × 10² a | 6.0 × 10¹ a | 3.0 × 10² a |
| 10 | 3.9 × 10² a, b | 5.7 × 10² a | 5.4 b |
| 12 | 3.3 × 10¹ a | 3.0 × 10¹ a | 0 b |
| 14 | 1.8 a | 2.9 a | 9.4 × 10¹ a |
| 17 | 7.4 × 10¹ a | 9.3 a | 5.3 × 10¹ a |
| 19 | 1.9 × 10³ a | 2.6 × 10¹ a | 2.9 b |
| 23 | 4.5 × 10² a | 1.2 × 10² a | 1.1 × 10¹ a |
| 24 | 5.4 a | 2.0 a | 1.6 × 10² a, b |
| 26 | 6.3 × 10¹ a | 9.5 a | 1.5 a |
| 30 | 0 a | 0 a | 1.8 a |

Treatment effect P = 0.02
Days effect P < 0.01
Treatment by Days Interaction P = 0.6
a, b Means not sharing the same letters differ at P < 0.1.

TABLE 2
Treatment Means.

| Treatment | Mean (CFU/g of faeces) | Significance |
|---|---|---|
| Control, | 1.9 × 10² | Control vs. 2 g DFM P = 0.06 |
| 0 g/day | | Control vs. 20 g DFM P = 0.01 |
| DFM, | 4.5 × 10¹ | 2 g DFM vs. 20 g DFM P = |
| 2 g/day | | 0.54 |
| DFM, | 2.8 × 10¹ | 20 g DFM vs. 200 g DFM P = |
| 20 g/day | | 0.01 |

P-value (treatment effect) = 0.02.

TABLE 3
Effects of Direct-fed microbials (DFM) on fecal shedding of
Nalidixic acid-resistant E. coli O157:H7 in cattle.

| | | DFM | | | |
|---|---|---|---|---|---|
| Day | Control | 2 gX | DFM 20 g | TC 2 g | TC 20 g |
| D0 | 3700000 | 3700000 | 3700000 | 3700000 | 3700000 |
| D1 | 3400000 | 120000 | 2400000 | 750000 | 3700000 |
| D3 | 900000 | 120000 | 580000 | 100000 | 580000 |
| D5 | 83000 | 33000 | 250000 | 60000 | 3800000 |
| D8 | 5000 | 470 | 14000 | 160 | 35000 |
| D10 | 2800 | 120 | 72 | 1300 | 520 |
| D12 | 1400 | 110 | 100 | 11000 | 570 |
| D15 | 2000 | 130 | 7 | 120 | 620 |
| D19 | 120 | 350 | 19 | 1700 | 17 |
| D22 | 17 | 17 | 21 | 18 | 9 |
| D24 | 230 | 230 | 0 | 18 | 14 |
| D26 | 9 | 61 | 0 | 0 | 0 |

### Example 2

### Assay to Select Enterococcus Bacteria That are Tolerant to Preferred Conditions

A number of publicly available Enterococcus bacteria were tested in the assay described below. The strains SF-273 (CHCC 4202) and SF-301 (CHCC 3828) were both tolerant to the below described testing conditions.

Exposure to Rumen Fluid

The medium of Bryant M P and Burkey L A (J. Dairy Science 1960) containing 40% rumen fluid (RF medium) was prepared either under 100% CO₂ (with sodium carbonate solution as buffer) or under 80%:20% N2:CO2 headspace gas (with sodium bicarbonate solution as buffer). This medium was used to score growth of the test strains as 0 (no growth) or +, ++, +++, or ++++(excellent growth) at 37 C after 48 to 72 h.

Exposure to Volatile Fatty Acids

Medium 10 of Bryant M P and Robinson I M (J. Dairy Science 1966) was prepared to test the tolerance of each test strain to volatile fatty acids (acetic, propionic and butyric acids, 200 to 400 mM). This medium was used to score growth of the test strains as 0 (no growth) or +, ++, +++, or ++++(excellent growth) at 37 C after 48 to 72 h. Only those strains having a +++ or ++++score advanced in the testing procedure are in present context an Enterococcus bacterium considered tolerant high concentrations of volatile fatty acids (200 to 400 mM mixtures of acetic, propionic and butyric acids).

Acid Tolerance

Medium 10 Bryant M P and Robinson I M (J. Dairy Science 1966) was prepared and poised at pH 4.0, 5.0, or 6.0 using a 5 N solution of HCl (hydrochloric acid). The medium was autoclaved, cooled and then inoculated with each test strain. These media were used to score growth of the test strains as 0 (no growth) or +, ++, +++, or ++++(excellent growth) at 37 C after 48 to 72 h. Only those strains having a +++ or ++++score in the pH 4.0 medium advanced in the testing procedure are in present context an Enterococcus bacterium considered tolerant of high acid (pH 4.0) concentrations.

Oxygen Tolerance

Medium 10 of Bryant M P and Robinson I M (J. Dairy Science 1966) was prepared to test the tolerance of each test strain to oxygen tolerance. In this test, the medium was modified as follows: 2% w/v agar was added to solidify the medium, the amount of resazurin (redox indicator) was doubled, and the amount of reductant, cysteine hydrochloride solution, was reduced by 50%. This medium was dispensed in 10 mL amounts and solidified after autoclaving in an upright position. Each test strain was grown in broth culture overnight and then stab-inoculated from top to bottom in the center of the 10 mL tubes of modified Medium 10. The inoculated tubes were exposed to the atmosphere for 5 min then closed and incubated overnight (ca. 18 h) at 37 C. After incubation the top one-centimeter of the medium had oxidized (turned the redox indicator pink) and the resultant growth pattern of each test strain was scored. The strain was scored as a strict anaerobe if visible growth along the stab line was only in the reduced portion of the tube. The strain was scored as an oxygen tolerant anaerobe if strain growth extended into the pink (oxidized) portion of the medium. The strain was scored as a facultative anaerobe if growth was visible along the stab line within both the reduced and oxidized portions of the tube. The strain was scored as an aerobe if growth was visible only within the oxidized portion of the medium. In the present context an Enterococcus bacterium is considered tolerant to complete anaerobiosis conditions if it grew in the reduced portion of the medium.

Oxygen Scavenging

Medium 10 as modified for oxygen tolerance testing was used. After oxygen tolerance capacity was scored, the stab-inoculated tubes were re-incubated at 37 C for an additional 24 h. Then the oxidized zone of each tube was examined and scored as follows. If the pink zone was re-reduced to colorless, then the strain was scored as being an oxygen scavenger (characteristic present or absent). In the present context an Enterococcus bacterium is considered an oxygen scavenger if it re-reduced the medium.

### Example 3

### Formulation of Farm Animal Product

Table 4 sets forth the composition of one embodiment of a farm animal product according to the present invention. In this embodiment depicted in Table 4, the farm animal product is not less than 200 billion CFU/gram. The farm animal product in Table 4 is packaged in 250 gm foil pouches, with 14 pouches per case, and it is available from Chr. Hansen, Inc. in Milwaukee, Wis. under the name Probios FS.

TABLE 4
Farm animal product containing probiotic Enterococcus bacteria.

| Ingredients | Typical Inclusion |
|---|---|
| Dried Enterococcus faecium Fermentation Product | (45.0-75.0%) |
| (50:50 mix of E. faecium strains SF-273 and SF-301) | |
| Sodium Thiosulfate | 0.12% |
| Sodium Aluminosilicate | 10.00% |
| Maltodextrin (Balance) | (44.88-14.88%) |
| Total | 100.00% |

The present invention is not to be limited in scope by the specific embodiments described herein. Indeed, various modifications of the present invention, in addition to those described herein, will be apparent to those of ordinary skill in the art from the foregoing description. Thus, such modifications are intended to fall within the scope of the following appended claims. Further, although the present invention has been described herein in the context of a particular implementation in a particular environment for a particular purpose, those of ordinary skill in the art will recognize that its usefulness is not limited thereto and that the present invention can be beneficially implemented in any number of environments for any number of purposes. Accordingly, the claims set forth below should be construed in view of the full breadth and spirit of the present invention as disclosed herein.

1. A farm animal product comprising at least 106 CFU/g of probiotic Enterococcus bacteria, wherein said product reduces the number of Escherichia coli O157:H7 cells quantified in faeces of challenged animals by at least 1.5 logs.

2. The farm animal product of claim 1, wherein said product reduces the number of Escherichia coli O157:H7 cells quantified in faeces of challenged animals by at least 2 logs.

3. The farm animal product of claim 1, wherein said product comprises Lactobacillus acidophilus, and wherein said product comprises at least 2.5 times more of said Enterococcus bacteria than of said Lactobacillus acidophilus bacteria measured as CFU/g.

4. The farm animal product of claim 3, wherein said product comprises at least 5000 times more of said Enterococcus bacteria than of said Lactobacillus acidophilus bacteria measured as CFU/g.

5. The farm animal product of claim 1, wherein said product comprises farm animal feedstuff ingredients.

6. The farm animal product of claim 1, wherein said probiotic Enterococcus bacteria are Enterococcus faecium bacteria.

7. The farm animal product of claim 1, wherein said product comprises at least 106 CFU/g of said probiotic Enterococcus bacteria.

8. The farm animal product of claim 1, wherein said Enterococcus bacteria are tolerant to conditions of high acid (pH 4.0), high concentrations of volatile fatty acids (200 to 400 mM mixtures of acetic, propionic and butyric acids) or complete anaerobiosis.

9. The farm animal product of claim 1, which consists essentially of Enterococcus bacteria.

10. The farm animal product of claim 1 which consists of Enterococcus bacteria.

11. The farm animal product of claim 1 which consists of at least two strains of Enterococcus bacteria.

12. A method for feeding a farm animal comprising feeding said farm animal with said farm animal product as in any one of claims 1-11.

13. The method of claim 12, wherein said farm animals are cattle.

14. The method of claim 12, wherein said farm animals are fed with an amount of said farm animal product that provides from 10⁷ CFU to 10¹² CFU Enterococcus bacteria per animal per day.

15. The method of claim 12, wherein said farm animals are fed with said farm animal product at least once a day for at least 10 days.

16. The method of claim 15, wherein said farm animals are fed with said farm animal product at least once a day for at least 20 days.

17. A farm animal product consisting of about 50% Enterococcus faecium strain SF-273 and about 50% Enterococcus faecium strain SF-301 in a total amount of about 2×10 CFU/g, wherein said product reduces the number of Escherichia coli O157:H7 cells quantified in faeces of challenged animals by at least 1.5 logs, upon administration of the farm animal product to such animals.
